(19) United States
(12) Patent Application Publication
(10) Pub. No.: US 2014/0257163 A1
(43) Pub. Date: Sep. 11, 2014

(54) LOWER-LEG ORTHOSIS

(71) Applicants: Jörn Rittweger, Bad Honnef (DE); Michel Ducos, Bremen (DE); Tobias Weber, Koln (DE); Gert-Peter Brüggemann, Koln (DE)

(72) Inventors: Jörn Rittweger, Bad Honnef (DE); Michel Ducos, Bremen (DE); Tobias Weber, Koln (DE); Gert-Peter Brüggemann, Koln (DE)

(73) Assignees: DEUTSCHE SPORTHOCHSCHULE KOLN, Koln (DE); DEUTSCHES ZENTRUM FUR LUFT- UND RAUMFAHRT E.V., Koln (DE)

(21) Appl. No.: 14/344,778

(22) PCT Filed: Sep. 11, 2012
(86) PCT No.: PCT/EP2012/067730

§ 371 Date: May 1, 2014

(30) Foreign Application Priority Data
Sep. 14, 2011 (DE) 10 2011 082 700.5

(51) Int. Cl. A61F 5/01

(52) U.S. Cl. CPC A61F 5/0127
USPC 602/28

(57) ABSTRACT

A lower-leg orthosis includes a splint for immobilizing the muscles of the lower leg. The splint comprises a lower-leg cuff and of a foot rest connected to the lower-leg cuff. A foot device is arranged under the foot rest, and the foot rest is supported on the ground via this foot device. The foot device has a ground contact surface and an elastic energy reservoir, which is arranged between the foot rest and the ground contact surface. Torques can be transferred from the foot rest surface to the ground contact surface via the foot device.

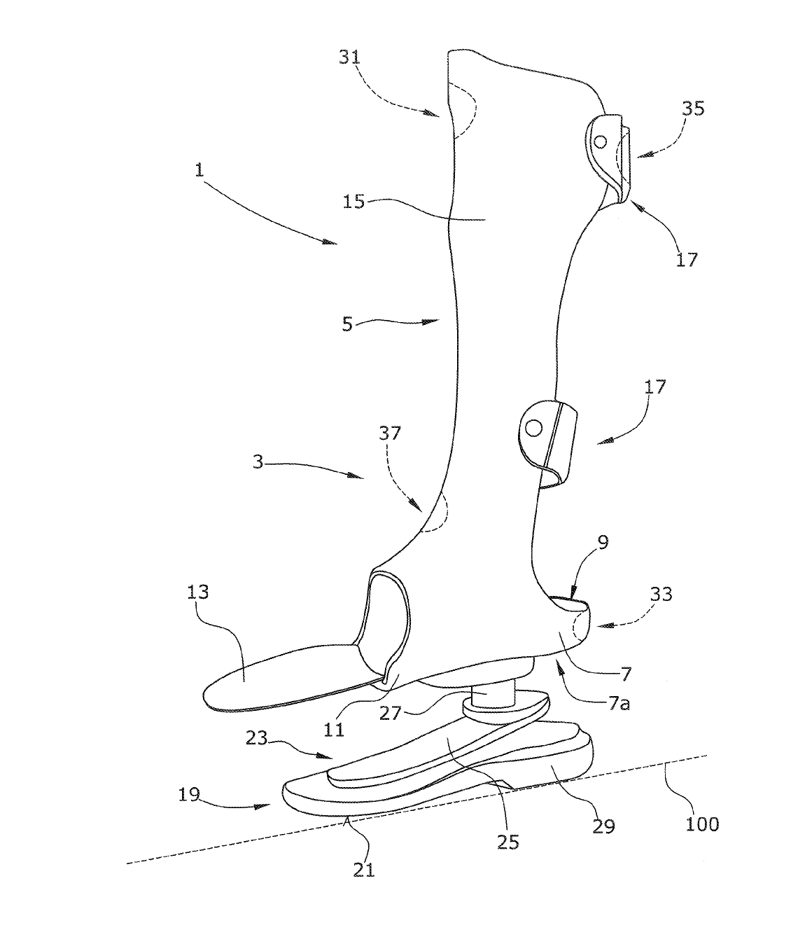

The present invention relates to a lower-leg orthosis with a splint for immobilizing the muscles of the lower leg, the splint being comprised of a lower-leg cuff and a foot rest.

Lower-leg orthoses are typically used in the context of injuries to the sinews and the ligamentous apparatus in the ankle joint. They serve to immobilize the lower leg to thereby prevent further damages and allow healing. Known lower-leg ortheses are based on the principle of restriction of mobility. Here, prior art lower-leg orthoses are comprised of a padded shell into which the foot and the lower leg of a patient are placed and which is fixed on the lower leg and the foot using closure straps. In this case, the shell rests against the calf area and under the foot. Corresponding ortheses are known, for example, under the designation “Malleo Immobil Walker” from the company Otto Bock.

The basic principle of such lower-leg ortheses lies in the mechanical restriction of mobility with respect to plantarflexion. When walking, the foot is rolled over a curved sole fixed to the shell of the orthosis.

However, it has been found that the gait pattern of a patient is affected by wearing such a lower-leg orthosis, since the mechanical processes occurring during normal walking, i.e. the generation of torques when rolling the foot, as well as a cyclical storage and release of elastic energy, are affected.

Further, the mechanical restriction of mobility caused by known ortheses does not automatically result in an immobilization of the muscles of the lower leg. The lower-leg orthosis still allows muscle contractions. A successful treatment, which requires an immobilization of the muscles, thus depends largely on the cooperation of the patient.

Moreover, owing to the disturbance in gait while wearing a conventional lower-leg orthosis, the walking speed of a patient is reduced, resulting in an increase in time and energy requirements. Thus, wearing a conventional lower-leg orthosis is perceived as uncomfortable.

Thus, it is an object of the present invention to provide a lower-leg orthosis which allows for a better immobilization of the muscles of the lower leg and which further improves the gait pattern of a patient. The lower-leg orthosis of the present invention is defined in claim 1.

The invention provides a lower-leg orthosis with a splint for the immobilization of the muscles in the lower leg, the splint being comprised of a lower-leg cuff and a foot rest connected with the lower-leg cuff. The invention is characterized by a foot device arranged below the foot rest. While walking, the foot rest is supported on the ground via this foot device. The foot device comprises a ground contact surface and an elastic energy reservoir. The elastic energy reservoir is arranged between the foot rest and the ground contact surface. Moreover, torques can be transferred from the foot rest onto the ground contact surface.

Owing to the foot device arranged below the foot rest and having an elastic energy reservoir, a gait pattern is achieved that is more harmonic than with the known lower-leg orthoses, since a cyclical storage and release of energy can occur via the elastic energy reservoir, leading to the more harmonic gait pattern. Since the foot rest is not supported directly on the ground, but the support is achieved via the foot device, the ground is no longer available as a bearing to a patient's forefoot so that a force development by the muscles of the lower leg can be reduced or prevented.

Thus, the lower-leg orthosis of the present invention is based on a basic principle different from conventional ortheses. While a conventional orthosis attempts to prevent a movement of the foot, the orthosis of the present invention prevents the development of force by the muscles in the lower leg. Thus, the forefoot of the patient is relieved and does not have to be fixed as with conventional orthoses. Therefore, it is neither necessary to provide a resistance against a plantarflexion of the foot by the orthosis of the present invention.

Moreover, the elastic energy reservoir allows for a buffering of energy while walking so that a harmonic gait pattern with a lower energy consumption is achieved.

With conventional orthoses, the calf muscles generate a torque as the ground reaction force, the force being introduced into the ground via the forefoot. In contrast therewith, the orthosis of the present invention allows the generation of torques, e.g. via the body weight, wherein the torque can be generated by an exertion of force via the shinbone into the lower-leg cuff and is transferred from the foot rest to the ground contact surface via the foot device in order to induce the ground reaction force.

The invention advantageously provides that an elastic energy reservoir is connected with the foot rest via a flange. The flange is preferably arranged concentrically with respect to the lower leg of the patient. This is advantageous in that the weight force of the body of the patient is induced linearly into the foot device via the lower leg. The flange further allows the release of the foot device from the foot rest so that the foot device can be exchanged.

The elastic energy reservoir can comprise a first foot plate that connects the ground contact surface with the flange. It may be provided that the first foot plate is elastic. It has been found that such an elastic first foot plate can advantageously serve as an elastic energy reservoir. In this regard, it is specifically provided that the first foot plate is a leaf spring.

In addition or as an alternative it may be provided that the resilient energy reservoir includes an elastic joint via which the first foot plate is connected with the flange. Thus, the storing of energy during walking can be realized in the elastic joint.

The storage of energy in the elastic joint and/or the elastically designed first foot plate advantageously allows for a cyclic storage of energy during walking owing to the fact that the weight force of a patient's body exerts a force on the elastic energy reservoir, whereby a potential energy can be stored in the elastic energy reservoir. After the rolling of the foot device via the ground contact surface, the elastic energy reservoir is relieved so that the potential energy is released and is returned when the foot device is pushed from the ground, thereby resulting in a harmonic gait. This makes it possible to walk with the orthosis of the invention in an energy-saving manner.

The elastically designed foot plate can also be rigidly connected with the flange so that the energy storage is effected exclusively in the first foot plate.

In one embodiment of the invention it is provided that the foot device comprises a second foot plate connected with the elastic energy reservoir, wherein the second foot plate comprises the ground contact surface. Providing a second foot plate allows for a substantial enlargement of the ground contact surface, and it is possible to realize anti-slip characteristics, for example. For instance, the second foot plate can be designed as a shoe sole. Specifically, it may be provided that the first foot plate extends under an acute angle with respect to the second foot plate.

The invention advantageously provides that the lower-leg cuff comprises a shell which, in use, rests against the shinbone. In this context, the shell may be adapted to the lower leg of a respective patient. The force provided by the body weight can advantageously be induced into the lower-leg prosthesis via the shell of the lower-leg cuff, whereby the torques are generated that are to be transferred onto the ground contact surface.

The foot rest can comprises a heel abutment and a metatarsus rest.

The shell and the foot rest may be integrally formed, whereby a particularly good adaptation of the lower-leg orthosis to the lower leg and the foot of a patient can be achieved.

It may be provided that the shell and/or the foot rest are made of a fiberglass material. This allows for a very strong and very light design of the present orthosis.

In one embodiment of the invention it is provided that the lower-leg cuff comprises a fastening device by which the lower-leg cuff can be fastened to the lower leg of a patient. The fastening device may be in the form of straps, for instance.

The following is a detailed description of the invention with reference to the FIGURE hereunder.

The sole FIGURE is a schematic perspective view of a lower-leg orthosis 1 of the present invention. The lower leg and the foot of a patient are not illustrated in the FIGURE. The lower-leg orthosis 1 has a splint 3 for the immobilization of the muscles in the lower leg of a patient, the splint 3 being comprised of a lower-leg cuff 5 and a foot rest 7 connected with the lower-leg cuff 5.

The foot rest 7 is formed integrally with the lower-leg cuff 5. The foot rest 7 is comprised of a heel abutment 9 and a metatarsus rest 11. Further, the foot rest 7 can include a forefoot plate 13 with a low flexural rigidity. The latter serves as a rest for the forefoot and reduces friction and resulting skin irritations in the sole of a patient's foot.

The lower-leg cuff 5 has a shell 15 resting against the lower leg of a patient. The shell 15 is conformed to the lower leg of a patient. The lower-leg cuff 5 is provided with a fastening device 17 formed by two fastening means, such as fastenings straps, fixed to the shell 15. The lower-leg cuff 5 can be fastened on the lower keg of a patient by means of the fastening device 17.

At the underside 7a of the foot rest 7, a foot device 19 is arranged via which the foot rest 7 can be supported on the ground 100. The foot device 19 comprises a ground contact surface 21 and an elastic energy reservoir 23. The elastic energy reservoir 23 is formed by a first foot plate 25 of elastic design, e.g. in the form of a leaf spring. The first foot plate 25 is connected with the foot rest 7 via a flange 27. The flange 27 is arranged below the lower leg of a patient, preferably concentrically with the lower leg, so that the weight force is induced linearly into the foot device 19 via the lower leg. The foot device 19 can be detached from the lower-leg cuff 5 via the flange 27 so that the foot device 19 can be exchanged or be used with other lower-leg cuffs 5. The lower-leg cuff 5 is adapted to the lower leg of a patient so that the foot device 19 can be provided as a standardized part that is connected with the lower-leg cuff 5 via the flange 27.

The foot device 19 further includes a second foot plate 29 connected with the first foot plate 25. In the embodiment illustrated in the FIGURE, the second foot plate 29 comprises the ground contact surface 21 and is designed in the manner of a shoe sole. The first foot plate 25 extends under an acute angle with respect to the second foot plate 29. The second foot plate 29 may have anti-slip characteristics, for example.

The lower-leg orthosis 1 allows for an improved immobilization of the lower leg of a patient, since the development of force in the lower leg muscles is prevented. Owing to the low flexural rigidity of the forefoot plate 13, the forefoot thus has no or only a negligible abutment so that a development of force in the lower leg upon a movement of the forefoot is prevented.

Further, while walking, the weight force of a patient is induced into the elastic energy reservoir 23 so that the same stores a part of the weight force as potential energy. In the embodiment illustrated in the FIGURE, this is achieved by a bending of the elastic first foot plate 25. The storage of energy occurs during the rolling process of the foot device 19 modeled on the rolling of a foot during normal movement. For this purpose, a torque is transferred from the foot rest 7 onto the foot device 19. The torque is generated by the body weight of a patient and is induced into the lower-leg cuff 5 from the shinbone. For this purpose, the shinbone has to rest on the lower-leg cuff 5 at at least a first contact point 31 schematically indicated in the FIGURE. Further, it is advantageous to provide a second contact point 33 at the heel abutment 9 of the foot rest 7. Via at least these two contact points 31, 33, the torque is generated and induced into the foot device 19 and transferred to the ground contact surface 21 for the generation of a ground reaction force. The transfer of torques causes an elastic bending of the first foot plate 25, whereby it becomes possible to store potential energy. As the foot is rolled via the second ground plate 29, the potential energy is released again and is returned into the system so that an energy-efficient and harmonic gait is made possible.

Upon setting the foot device 19 on the ground 100, the lower leg of the patient is supported at the lower-leg cuff 5 at a third contact point 35 situated in the upper portion of the fastening device 17 and at a fourth contact point 37 in the lower portion of the shinbone.

Advantageously, the torques are transferred from the lower leg of the patient to the lower-leg orthosis 1 of the invention, when the distance between the first contact point 31 and the second contact point 33 or the third contact point 35 and the fourth contact point 37 is as large as possible. Further, the contact points should rest on the lower leg of the patient with an area as large as possible in order to avoid skin irritations and pressure points.

The orthosis 1 of the present invention is applicable in particular in the treatment of serial and parallel structures of the lower leg, namely the ligaments, sinews, capsules, muscles, or in cases of broken bones in the foot region. The orthosis can also be used in early mobilization after a complete immobilization, for example after an injury or surgery, or it may be used in cases of paralysis- or age-related disturbances in gait. The lower-leg orthosis 1 of the invention specifically allows for a relief of the lower leg muscles in their function during the gait cycle.

In the embodiment illustrated in the FIGURE, the first foot plate 25 is rigidly connected with the flange 27. Of course, it is also possible to connect the first foot plate 25 with the flange via an elastically designed hinge joint, e.g. torsionally flexible spring. In this case, the first foot plate 25 can be configured so as to be elastic or also such that it has a high flexural rigidity.

The elastic energy reservoir may have a variable rigidity. Thereby, the lower-leg orthosis of the invention can be adapted, for example, to the weight of a patient or to the walking speed and the running speed.

In a particularly advantageous embodiment, the lower-leg orthosis allows for a harmonic gait of the patient, since it enables the cyclic energy storage and energy return during a gait cycle. Of course, the height difference caused by the foot device 19 must be compensated at the healthy leg of a patient, e.g. by providing for a greater thickness of the shoe sole.

Moreover, the immobilization of the lower leg muscles is significantly improved, since the force development in the muscles is prevented almost completely. Thereby, the treatment is improved considerably, and the more harmonic gait achieved makes wearing the lower-leg orthosis more comfortable for the patient.

1-15. (canceled)

16. A lower-leg orthosis with a splint for the immobilization of the muscles of the lower leg,
the splint including a lower-leg cuff and a foot rest connected with the lower-leg cuff, and comprising:
a foot device arranged below the foot rest, via which the foot rest is supported on the ground,
the foot device comprising a ground contact surface and an elastic energy reservoir arranged between the foot rest and the ground contact surface, and wherein torques are transferable from the foot rest to the ground contact surface via the foot device.

17. The lower-leg orthosis of claim 16, wherein the elastic energy reservoir is connected with the foot rest via a flange.

18. The lower-leg orthosis of claim 16, wherein the elastic energy reservoir comprises a first foot plate connecting the ground contact surface with the flange.

19. The lower-leg orthosis of claim 18, wherein the first foot plate is elastic.

20. The lower-leg orthosis of claim 19, wherein the first foot plate is a leaf spring.

21. The lower-leg orthosis of claim 18, wherein the elastic energy reservoir comprises an elastic joint via which the first foot plate is connected with the flange.

22. The lower-leg orthosis of claim 18, wherein the first foot plate is rigidly connected with the flange.

23. The lower-leg orthosis of claim 16, wherein the foot device comprises a second foot plate connected with the elastic energy reservoir, the second foot plate comprising the ground contact surface.

24. The lower-leg orthosis of claim 23, wherein the second foot plate is configured as a shoe sole.

25. The lower-leg orthosis of claim 16, wherein the lower-leg cuff comprises a shell resting on a shinbone of the lower leg.

26. The lower-leg orthosis of claim 25, wherein the shell is adapted to the lower leg.

27. The lower-leg orthosis of claim 16, wherein the foot rest comprises a heel abutment and a metatarsus rest.

28. The lower-leg orthosis of claim 25, wherein the shell and the foot rest are formed integrally.

29. The lower-leg orthosis of claim 16, wherein the shell and the foot rest are made of a fiberglass material.

30. The lower-leg orthosis of claim 16, wherein the lower-leg cuff comprises a fastening device arranged to fasten the lower-leg cuff on the lower leg.
